(19) United States
(12) Patent Application Publication
(10) Pub. No.: US 2026/0240743 A1
(43) Pub. Date: Aug. 20, 2026

(54) ANTI-INFLAMMATORY AND ANTI-AGING COSMETIC FOR USE AFTER MEDICAL AESTHETIC PROCEDURE

(71) Applicants: HANGZHOU PAITAI BIOCHEMICAL TECHNOLOGY CO., LTD, Hangzhou (CN); SHANGHAI PAITI BIOTECHNOLOGY CO., LTD, Shanghai (CN)

(72) Inventors: Minhua HONG, Hangzhou (CN); Haiying XING, Hangzhou (CN); Xinyou YU, Hangzhou (CN); Xinru CHEN, Hangzhou (CN); Chunyang JIANG, Hangzhou (CN); Diqing WANG, Hangzhou (CN); Xianglong ZHAO, Hangzhou (CN); Rong TANG, Hangzhou (CN)

(73) Assignees: HANGZHOU PAITAI BIOCHEMICAL TECHNOLOGY CO., LTD, Hangzhou (CN); SHANGHAI PAITI BIOTECHNOLOGY CO., LTD, Shanghai (CN)

(21) Appl. No.: 19/647,563

(22) Filed: Apr. 14, 2026

Related U.S. Application Data
(63) Continuation of application No. PCT/CN2026/081257, filed on Mar. 4, 2026.

(30) Foreign Application Priority Data
Mar. 28, 2025 (CN) 202510377693.4

(51) Int. Cl. A61K 8/64; A61K 8/14; A61K 8/34; A61K 8/35; A61K 8/362; A61K 8/67; A61K 8/73; A61K 8/81; A61K 8/99; A61Q 19/00; A61Q 19/08

(52) U.S. Cl. CPC A61K 8/64; A61K 8/14; A61K 8/345; A61K 8/35; A61K 8/362; A61K 8/671; A61K 8/676; A61K 8/678; A61K 8/733; A61K 8/8147; A61K 8/99; A61Q 19/005; A61Q 19/08; A61K 2800/48; A61K 2800/524; A61K 2800/5922; A61K 2800/81; A61K 2800/85

(57) ABSTRACT

An anti-inflammatory and anti-aging cosmetic for use after a medical aesthetic procedure is prepared from an active ingredient, an excipient, and water, and can be used in cosmetics. The excipient further includes a thickener, a humectant, and a preservative. The active ingredient includes at least an active substance that includes at least one of acetyl hexapeptide-8, oligopeptide-1, dipeptide diaminobutyroyl benzylamide diacetate, carnosine, palmitoyl tripeptide-1, ACE liposome, and bifida ferment lysate. An active agent may further be added to the active ingredient. The liquid composite has the advantages of no cytotoxicity, good safety, promotion of cell proliferation, excellent anti-inflammatory effect, effective anti-aging properties, no irritation to skin, and superior anti-inflammatory effect after medical aesthetic procedures.

### CROSS-REFERENCE TO RELATED APPLICATIONS

This application is a continuation of International Patent Application No. PCT/CN2026/081257 with a filing date of Mar. 4, 2026, designating the United States, now pending, and further claims priority to Chinese Patent Application No. 202510377693.4 with a filing date of Mar. 28, 2025. The content of the aforementioned applications, including any intervening amendments thereto, is incorporated herein by reference.

### FIELD OF THE INVENTION

The present disclosure belongs to the field of cosmetics, and particularly relates to an anti-inflammatory and anti-aging cosmetic for use after a medical aesthetic procedure.

### BACKGROUND OF THE INVENTION

With people's growing pursuit of beauty, the field of medical aesthetics has encountered unprecedented opportunities for development. Various minimally invasive aesthetic techniques have emerged in great numbers. Among them, techniques such as mesotherapy (e.g., water light needle hydrating injection) and photoelectric aesthetic treatments are highly favored due to their remarkable effects. Through different mechanisms of action, these techniques effectively address a series of skin problems such as pigmentation, aging, acne, and dryness, rejuvenating the skin. However, minimally invasive medical aesthetic procedures will inevitably have a certain impact on the skin barrier while bringing beauty. The skin barrier, as an important protective layer of the human body, once damaged, not only affects the healthy state of the skin but may also lead to various skin problems. For some patients with sensitive skin, the harm caused by the release of inflammatory cytokines during the repair process can be more persistent. Therefore, using barrier repair products has become an indispensable part of care after minimally invasive medical aesthetic procedures. Moreover, inflamed skin is more susceptible to external stimuli than normal skin. The external stimuli can induce inflammation and release free radicals that participate in inflammatory and glycation processes, forming a vicious cycle of inflammation-glycation-oxidation. Consequently, this leads to more rapid skin aging. Hence, the anti-aging effect for inflamed skin is as important as anti-inflammatory repair. However, class II dressings commonly used in the past after medical aesthetic procedures mainly function by forming a protective layer on the wound surface, thereby acting as a physical barrier. CN117085174A discloses a liquid dressing for wound care after medical aesthetic procedures and a preparation method thereof. However, the class II dressings lack pharmacological efficacy and cannot be degraded and absorbed. Their use is restricted to large-molecule moisturizing substances, which cannot be truly absorbed through the skin. Once the skin wound heals rapidly, the therapeutic effect of the class II dressings diminishes significantly. Conversely, for conventional cosmetics with anti-aging efficacy, there is often a lack of research on individuals in the inflammatory stage following medical aesthetic procedures.

The repair process after medical aesthetic procedures may be divided into three stages. The first is the acute inflammatory stage, occurring within 24 hours after an operation. Within minutes of tissue injury, immune cells rapidly infiltrate the wound. Among these, neutrophils and macrophages are the primary effector cells during the inflammatory stage. These immune cells release various cytokines and chemokines, recruiting cells from the blood and wound margins into the local wound site. Meanwhile, the secreted cytokines can further promote cell proliferation at the injury site. Furthermore, immune cells secrete a plurality of proteases and reactive oxygen species (ROS) to defend against pathogenic microbial invasion and clear damaged cells. Although the early inflammatory response is beneficial for wound healing, numerous studies have shown that an increase in the number of immune cells at the injury site or a prolonged inflammatory response will disrupt the wound repair process, ultimately leading to non-healing wounds or pathological scar formation. This is primarily due to the continuous production of ROS at the injury site, which damages cell structure and function, disrupting the normal skin repair process. Approximately 2 to 10 days after skin injury, new tissue begins to form, making the repair process enter into the proliferative stage. This stage mainly involves angiogenesis, granulation tissue formation, and re-epithelialization. Angiogenesis is crucial for wound repair. Macrophages and injured endothelial cells release FGF-2 and VEGF, promoting angiogenesis. Subsequently, capillary sprouts infiltrate the injury site, forming granulation tissue along with fibroblasts and immune cells, which provides nutrients and oxygen for cellular metabolism. Finally, growth factors such as EGF and TGF-β stimulate the proliferation and migration of keratinocytes at the wound edges, achieving re-epithelialization of the injured epidermis. Following the completion of re-epithelialization, keratinocytes differentiate, restoring the epidermal barrier function. Meanwhile, fibroblasts in the dermis proliferate and migrate to the injury site, producing a substantial amount of extracellular matrix. At the end of this stage, growth factors (TGF-β) or mechanical stress stimulate some fibroblasts to differentiate into myofibroblasts, which express α-smooth muscle actin to promote wound contraction. The third is the tissue restructuring stage, which typically begins 2 to 3 weeks after injury and can last for years. During this stage, the signals activated by the injury gradually diminish. Once wound re-epithelialization is completed, keratinocytes cease proliferation and migration and begin to differentiate. Blood vessels within the newly formed tissue gradually mature, forming a functional network. Most endothelial cells, inflammatory cells, fibroblasts, and myofibroblasts undergo apoptosis or withdraw from the wound site. The granulation tissue formed during the early repair stage is gradually replaced by newly formed dermal matrix rich in collagen. It is noteworthy that normal skin tissue is primarily composed of type III collagen, forming a basket-weave structure, whereas the injury site is reconstructed into thicker type I collagen, forming a dense parallel structure. Epidermal appendages, such as hair follicles and sebaceous glands, generally cannot regenerate during this process. Therefore, the newly formed tissue often differs from healthy tissue, manifesting as a scar mainly characterized by decreased elasticity and tensile strength.

### SUMMARY OF THE INVENTION

The present disclosure, considering the application scenarios and functional limitations of both class II dressings and conventional cosmetics, proposes the concept of a cosmeceutical-device combination. Specifically, a sterile class II dressing product is used during the acute inflammatory stage after a medical aesthetic procedure to inhibit the onset of acute inflammation and promote wound healing. This is subsequently paired with a mild and soothing anti-aging efficacy product to meet anti-aging needs.

An objective of the present disclosure is to provide an anti-inflammatory and anti-aging cosmetic for use after a medical aesthetic procedure that has the advantages of no cytotoxicity, good safety, promotion of cell proliferation, good anti-inflammatory effect, good anti-aging effect, no irritation to skin, and good anti-inflammatory effect after medical aesthetic procedures.

The technical solutions adopted by the present disclosure to achieve the above objective are as follows.

A liquid composite includes an active ingredient and water, where a content of the active ingredient in the liquid composite is 0.003-6.0 wt %; and the active ingredient includes an active substance that includes at least one of acetyl hexapeptide-8, oligopeptide-1, dipeptide diaminobutyroyl benzylamide diacetate, carnosine, palmitoyl tripeptide-1, palmitoyl tripeptide-5, ACE liposome, and bifida ferment lysate. The liquid composite of the present disclosure is a product that has both anti-inflammatory and anti-aging effects and can be used in daily skin care and after medical aesthetic procedures. It can not only make up for the anti-aging effect of class II dressings, but also meet the mild anti-inflammatory effect of cosmetics. The formulation is simple in composition, and the process operations are simple and convenient.

Preferably, the liquid composite further includes a thickener that includes at least one of carbomer, xanthan gum, and sclerotium gum, where a content of the thickener in the liquid composite is 0.01-1.0 wt %.

Preferably, the liquid composite further includes a humectant that includes at least one of glycerin, 1,2-pentanediol, 1,2-hexanediol, dipropylene glycol, carboxymethyl cellulose, and chitin, where a content of the humectant in the liquid composite is 1-20 wt %.

Preferably, the liquid composite further includes a preservative that is a raspberry ketone system, where a content of the preservative in the liquid composite is 0.1-1.0 wt %.

Preferably, the active ingredient further includes an active agent that includes a succinate derivative, where the succinate derivative is prepared from 2-aminoethyl hydrogen sulfate and a maleate, and the maleate is prepared from trimethylolpropane and maleic anhydride. It has been found in the present disclosure that succinate derivatives can be co-formulated with active substances to enhance the anti-aging and anti-inflammatory effects of the resulting liquid composite in the presence of other excipients. The liquid composite has no cytotoxicity and no irritation to skin, and has good safety.

More preferably, a content of the active agent in the liquid composite is 0.003-1.0 wt %; or
a content of the active substance in the liquid composite is 0.003-5.0 wt %.

More preferably, the maleate is prepared by: mixing trimethylolpropane and maleic anhydride for reacting to obtain the maleate, where the trimethylolpropane is used in an amount of 50-150 wt % of the maleic anhydride.

More preferably, the succinate derivative is prepared by: mixing 2-aminoethyl hydrogen sulfate and a maleate in an aqueous ethanol solution containing sodium hydroxide for reacting to obtain the succinate derivative, where the 2-aminoethyl hydrogen sulfate is used in an amount of 20-30 wt % of the maleate.

The present disclosure discloses use of the liquid composite described above in preparing an anti-inflammatory cosmetic and/or an anti-aging cosmetic and/or a non-irritating product.

Preferably, the maleate is prepared by: adding trimethylolpropane to maleic anhydride at 60-80° C., and reacting at 85-95° C. for 2-8 h; and after the reaction is completed, obtaining the maleate.

More preferably, in the preparation of the maleate, the trimethylolpropane is used in an amount of 50-150 wt % of the maleic anhydride.

Preferably, the succinate derivative is prepared by: adding sodium hydroxide and 2-aminoethyl hydrogen sulfate to deionized water, and mixing with stirring; then adding a maleate and ethanol at 30-50° C. and reacting for 2-8 h; after the reaction is completed, cooling to form a precipitate; and carrying out filtration, washing, and distillation under reduced pressure to obtain the succinate derivative.

More preferably, in the preparation of the succinate derivative, the sodium hydroxide is used in an amount of 30-50 wt % of the deionized water.

More preferably, in the preparation of the succinate derivative, the 2-aminoethyl hydrogen sulfate is used in an amount of 40-60 wt % of the deionized water.

More preferably, in the preparation of the succinate derivative, the 2-aminoethyl hydrogen sulfate is used in an amount of 20-30 wt % of the maleate.

More preferably, in the preparation of the succinate derivative, absolute ethanol is used in an amount of 100-200 wt % of the maleate.

The present disclosure provides a preparation method for a liquid composite, including: adding a thickener to water, then adding a humectant and a preservative, and homogenizing at 70-90° C. for 2-10 min; after the completion of homogenization, cooling down to 20-50° C., then adding an active ingredient, mixing well with stirring, and cooling down to room temperature to obtain the liquid composite.

Preferably, the active ingredient includes an active substance that includes at least one of acetyl hexapeptide-8, oligopeptide-1, dipeptide diaminobutyroyl benzylamide diacetate, carnosine, palmitoyl tripeptide-1, ACE liposome, and bifida ferment lysate, where a content of the active substance in the liquid composite is 0.003-5.0 wt %.

Preferably, the thickener includes at least one of carbomer, xanthan gum, and sclerotium gum, and a content of the thickener in the liquid composite is 0.01-1.0 wt %.

Preferably, the humectant includes at least one of glycerin, 1,2-pentanediol, dipropylene glycol, carboxymethyl cellulose, and chitin, and a content of the humectant in the liquid composite is 1-20 wt %.

Preferably, the preservative is a raspberry ketone system, and a content of the preservative in the liquid composite is 0.1-1.0 wt %.

Preferably, the balance of the liquid composite was water.

Preferably, the active ingredient further includes an active agent, and a content of the active agent in the liquid composite is 0.003-1.0 wt %. The active agent further includes a succinate derivative and/or propane-1,2-diol alginate. In the present disclosure, the propane-1,2-diol alginate may alternatively be added as the active agent. The propane-1,2-diol alginate and the succinate derivatives may be used as the active agents. Under the action of the active substance and the excipients, the resulting liquid composite has better anti-aging and anti-inflammatory effects, has no cytotoxicity and no irritation to skin, and has good safety.

More preferably, the active agent includes a succinate derivative, and a content of the succinate derivative in the liquid composite is 0.003-0.5 wt %.

More preferably, the active agent further includes propane-1,2-diol alginate, and a content of the propane-1,2-diol alginate in the liquid composite is 0.003-0.5 wt %.

Reagents which are harmless to the human body, such as ethylenediamine tetraacetic acid (EDTA), EDTA disodium, and an amino acid, can further be added in the present disclosure without affecting the effects of the present disclosure. The amino acid includes amino acids such as arginine.

In the present disclosure, the liquid composite prepared from an active ingredient, an excipient, and water can be used in cosmetics. The excipient further includes a thickener, a humectant, and a preservative. The active ingredient in the present disclosure includes at least an active substance that includes at least one of acetyl hexapeptide-8, oligopeptide-1, dipeptide diaminobutyroyl benzylamide diacetate, carnosine, palmitoyl tripeptide-1, ACE liposome, and bifida ferment lysate. The liquid composite including the above active substance has excellent anti-inflammatory and anti-aging effects. Further, the active agent may be further added in the present disclosure. With the active agent, under the action of the active substance and the excipients, the resulting liquid composite has more excellent effects. The liquid composite has the advantages of no cytotoxicity, good safety, promotion of cell proliferation, good anti-inflammatory effect, good anti-aging effect, no irritation to skin, and good anti-inflammatory effect after medical aesthetic procedures. Therefore, the present disclosure provides an anti-inflammatory and anti-aging cosmetic for use after a medical aesthetic procedure that has the advantages of no cytotoxicity, good safety, promotion of cell proliferation, good anti-inflammatory effect, good anti-aging effect, no irritation to skin, and good anti-inflammatory effect after medical aesthetic procedures.

### BRIEF DESCRIPTION OF THE DRAWINGS

FIG. 1 is a graph showing the results of the expression of inflammatory cytokines;

FIG. 2 is a graph showing the results of the expression of inflammatory cytokines;

FIG. 3 is a graph showing the results of the expression of inflammatory cytokines;

FIG. 4 is a graph showing the results of the expression of inflammatory cytokines;

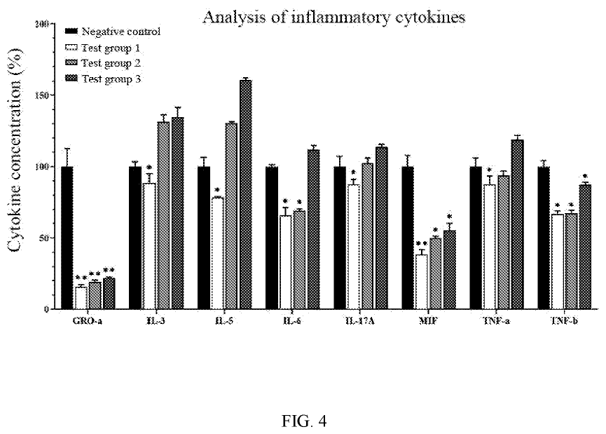

FIG. 5 is a graph showing the expression of inflammatory cytokines of macrophage migration inhibitory factor (MIF);

FIG. 6 is a graph showing the results of relative expression of genes;

FIG. 7 is a graph showing the results of relative expression of genes;

FIG. 8 is a graph showing the results of relative expression of genes;

FIG. 9 is a graph showing the results of relative expression of genes; and

FIG. 10 is a graph showing relative expression of gene NFE2L2.

### DETAILED DESCRIPTION OF THE EMBODIMENTS

The technical solutions of the embodiments of the present disclosure are clearly and completely described below with reference to the accompanying drawings. Apparently, the described embodiments are merely a part rather than all of the embodiments of the present disclosure. All other embodiments obtained by a person of ordinary skill in the art on the basis of the embodiments of the present invention without creative efforts shall fall within the protection scope of the present invention.

The concepts involved in the present disclosure will first be described below with reference to the accompanying drawings. It should be noted here that the following descriptions of the various concepts are only intended to make the content of the present disclosure easier to understand, and are not meant to limit the protection scope of the present disclosure. Moreover, provided that there is no conflict, the embodiments of the present disclosure and the features in the embodiments may be combined with each other. The present disclosure will be described in detail below with reference to the drawings and embodiments.

### Example 1: Preparation Method for Liquid Composite

Preparation of a liquid composite: a thickener was added to water, and then a humectant and a preservative were added and homogenized at 80° C. for 3 min. After the completion of homogenization, the mixture was cooled down to 40° C., then added with an active ingredient, and mixed well with stirring. The resulting mixture was cooled down to room temperature to obtain the liquid composite. The thickener was carbomer. The humectant was glycerol. The preservative was a raspberry ketone system. The active ingredient included active substances, namely ACE liposome, acetyl hexapeptide-8, dipeptide diaminobutyroyl benzylamide diacetate, and bifida ferment lysate. The ACE liposome was Lips-014 ACE liposome. The content of the thickener in the liquid composite was 0.24 wt %. The content of the humectant in the liquid composite was 8 wt %. The content of the preservative in the liquid composite was 0.35 wt %. The content of the ACE liposome in the liquid composite was 1.5 wt %. The content of the acetyl hexapeptide-8 in the liquid composite was 0.005 wt %. The content of the dipeptide diaminobutyroyl benzylamide diacetate in the liquid composite was 0.01 wt %. The content of the bifida ferment lysate in the liquid composite was 1.5 wt %. The remaining reagent was water. The remaining reagent was water.

The liquid composite prepared in Example 1 included the active ingredient, the thickener, the humectant, the preservative, and water. The active ingredient included active substances, namely ACE liposome, acetyl hexapeptide-8, dipeptide diaminobutyroyl benzylamide diacetate, and bifida ferment lysate. The ACE liposome was Lips-014 ACE liposome. The content of the ACE liposome in the liquid composite was 1.5 wt %. The content of the acetyl hexapeptide-8 in the liquid composite was 0.005 wt %. The content of the dipeptide diaminobutyroyl benzylamide diacetate in the liquid composite was 0.01 wt %. The content of the bifida ferment lysate in the liquid composite was 1.5 wt %. The thickener was carbomer, and the content of the thickener in the liquid composite was 0.24 wt %. The humectant was glycerol, and the content of the humectant in the liquid composite was 8 wt %. The preservative was a raspberry ketone system, and the content of the preservative in the liquid composite was 0.35 wt %. The remaining reagent was water

### Example 2: Preparation Method for Liquid Composite

This example was different from Example 1 in that the active ingredient further included an active agent. The active agent was a succinate derivative. The content of the succinate derivative in the liquid composite was 0.009 wt %.

Preparation of a maleate: trimethylolpropane was added to maleic anhydride at 70° C. for reacting at 90° C. for 4 h. After the reaction was completed, the maleate was obtained. The trimethylolpropane was used in an amount of 100 wt % of the maleic anhydride.

Preparation of the succinate derivative: sodium hydroxide and 2-aminoethyl hydrogen sulfate were added to deionized water, and mixed with stirring. The maleate and ethanol were then added at 40° C. for reacting for 4 h. After the reaction was completed, a precipitate was formed by cooling, and then was filtered, washed, and distilled under reduced pressure to obtain the succinate derivative. The sodium hydroxide was used in an amount of 40 wt % of the deionized water. The 2-aminoethyl hydrogen sulfate was used in an amount of 50 wt % of the deionized water. The 2-aminoethyl hydrogen sulfate was used in an amount of 25 wt % of the maleate. Absolute ethanol was used in an amount of 150 wt % of the maleate.

The liquid composite prepared in Example 2 included the active ingredient, the thickener, the humectant, the preservative, and water. The active ingredient included active substances and the active agent. The active substances were ACE liposome, acetyl hexapeptide-8, dipeptide diaminobutyroyl benzylamide diacetate, and bifida ferment lysate. The ACE liposome was Lips-014 ACE liposome, and the content of the ACE liposome in the liquid composite was 1.5 wt %. The content of the acetyl hexapeptide-8 in the liquid composite was 0.005 wt %. The content of the dipeptide diaminobutyroyl benzylamide diacetate in the liquid composite was 0.01 wt %. The content of the bifida ferment lysate in the liquid composite was 1.5 wt %. The active agent was the succinate derivative, and the content of the succinate derivative in the liquid composite was 0.009 wt %. The thickener was carbomer, and the content of the thickener in the liquid composite was 0.24 wt %. The humectant was glycerol, and the content of the humectant in the liquid composite was 8 wt %. The preservative was a raspberry ketone system, and the content of the preservative in the liquid composite was 0.35 wt %. The remaining reagent was water.

### Example 3: Preparation Method for Liquid Composite

This example was different from Example 1 in that the active ingredient further included an active agent. The active agent was a succinate derivative. The content of the succinate derivative in the liquid composite was 0.03 wt %.

The liquid composite prepared in Example 3 included the active ingredient, the thickener, the humectant, the preservative, and water. The active ingredient included active substances and the active agent. The active substances were ACE liposome, acetyl hexapeptide-8, dipeptide diaminobutyroyl benzylamide diacetate, and bifida ferment lysate. The ACE liposome was Lips-014 ACE liposome, and the content of the ACE liposome in the liquid composite was 1.5 wt %. The content of the acetyl hexapeptide-8 in the liquid composite was 0.005 wt %. The content of the dipeptide diaminobutyroyl benzylamide diacetate in the liquid composite was 0.01 wt %. The content of the bifida ferment lysate in the liquid composite was 1.5 wt %. The active agent was the succinate derivative, and the content of the succinate derivative in the liquid composite was 0.03 wt %. The thickener was carbomer, and the content of the thickener in the liquid composite was 0.24 wt %. The humectant was glycerol, and the content of the humectant in the liquid composite was 8 wt %. The preservative was a raspberry ketone system, and the content of the preservative in the liquid composite was 0.35 wt %. The remaining reagent was water.

### Example 4: Preparation Method for Liquid Composite

This example was different from Example 3 in that the active agent further included propane-1,2-diol alginate. The content of the propane-1,2-diol alginate in the liquid composite was 0.003 wt %.

The liquid composite prepared in Example 4 included the active ingredient, the thickener, the humectant, the preservative, and water. The active ingredient included active substances and the active agent. The active substances were ACE liposome, acetyl hexapeptide-8, dipeptide diaminobutyroyl benzylamide diacetate, and bifida ferment lysate. The ACE liposome was Lips-014 ACE liposome, and the content of the ACE liposome in the liquid composite was 1.5 wt %. The content of the acetyl hexapeptide-8 in the liquid composite was 0.005 wt %. The content of the dipeptide diaminobutyroyl benzylamide diacetate in the liquid composite was 0.01 wt %. The content of the bifida ferment lysate in the liquid composite was 1.5 wt %. The active agent was the succinate derivative and the propane-1,2-diol alginate, and the content of the succinate derivative in the liquid composite was 0.03 wt %, while the content of the propane-1,2-diol alginate was 0.003 wt %. The thickener was carbomer, and the content of the thickener in the liquid composite was 0.24 wt %. The humectant was glycerol, and the content of the humectant in the liquid composite was 8 wt %. The preservative was a raspberry ketone system, and the content of the preservative in the liquid composite was 0.35 wt %. The remaining reagent was water.

### Example 5: Preparation Method for Liquid Composite

This example was different from Example 3 in that the active agent further included propane-1,2-diol alginate. The content of the propane-1,2-diol alginate in the liquid composite was 0.01 wt %.

The liquid composite prepared in Example 5 included the active ingredient, the thickener, the humectant, the preservative, and water. The active ingredient included active substances and the active agent. The active substances were ACE liposome, acetyl hexapeptide-8, dipeptide diaminobutyroyl benzylamide diacetate, and bifida ferment lysate. The ACE liposome was Lips-014 ACE liposome, and the content of the ACE liposome in the liquid composite was 1.5 wt %. The content of the acetyl hexapeptide-8 in the liquid composite was 0.005 wt %. The content of the dipeptide diaminobutyroyl benzylamide diacetate in the liquid composite was 0.01 wt %. The content of the bifida ferment lysate in the liquid composite was 1.5 wt %. The active agent was the succinate derivative and the propane-1,2-diol alginate, and the content of the succinate derivative in the liquid composite was 0.03 wt %, while the content of the propane-1,2-diol alginate was 0.01 wt %. The thickener was carbomer, and the content of the thickener in the liquid composite was 0.24 wt %. The humectant was glycerol, and the content of the humectant in the liquid composite was 8 wt %. The preservative was a raspberry ketone system, and the content of the preservative in the liquid composite was 0.35 wt %. The remaining reagent was water.

### Comparative Example 1: Preparation Method for Liquid Composite

This comparative example was different from Example 2 in that the content of the succinate derivative in the liquid composite was 0.001 wt %.

The liquid composite prepared in Comparative Example 1 included the active ingredient, the thickener, the humectant, the preservative, and water. The active ingredient included active substances and the active agent. The active substances were ACE liposome, acetyl hexapeptide-8, dipeptide diaminobutyroyl benzylamide diacetate, and bifida ferment lysate. The ACE liposome was Lips-014 ACE liposome, and the content of the ACE liposome in the liquid composite was 1.5 wt %. The content of the acetyl hexapeptide-8 in the liquid composite was 0.005 wt %. The content of the dipeptide diaminobutyroyl benzylamide diacetate in the liquid composite was 0.01 wt %. The content of the bifida ferment lysate in the liquid composite was 1.5 wt %. The active agent was the succinate derivative, and the content of the succinate derivative in the liquid composite was 0.001 wt %. The thickener was carbomer, and the content of the thickener in the liquid composite was 0.24 wt %. The humectant was glycerol, and the content of the humectant in the liquid composite was 8 wt %. The preservative was a raspberry ketone system, and the content of the preservative in the liquid composite was 0.35 wt %. The remaining reagent was water.

### Comparative Example 2: Preparation Method for Liquid Composite

This comparative example was different from Example 2 in that no active substance was added to the liquid composite.

The liquid composite prepared in Comparative Example 2 included the active ingredient, the thickener, the humectant, the preservative, and water. The active ingredient included an active agent. The active agent was a succinate derivative, and the content of the succinate derivative in the liquid composite was 0.009 wt %. The thickener was carbomer, and the content of the thickener in the liquid composite was 0.24 wt %. The humectant was glycerol, and the content of the humectant in the liquid composite was 8 wt %. The preservative was a raspberry ketone system, and the content of the preservative in the liquid composite was 0.35 wt %. The remaining reagent was water.

In the selection of the components of the liquid composite in the present disclosure, without affecting the technical effects of the present disclosure, glycerol, EDTA disodium, raspberry ketone, 1,2-hexanediol, xanthan gum, 1,2-pentanediol, carbomer, carboxymethyl chitosan, arginine, dipropylene glycol, ACE liposome, acetyl hexapeptide-8, dipeptide diaminobutyroyl benzylamide diacetate, oligopeptide-1, palmitoyl tripeptide-5, carnosine, and bifida ferment lysate can be selected arbitrarily.

### TEST EXAMPLES

### 1. Cytotoxicity Test

In the present disclosure, the liquid composite prepared in Example 1 was subjected to a cytotoxicity test. The liquid composite of Example 1 was mixed with a culture to prepare 8 liquid composite test groups with different volume percentages: 2.50%, 1.25%, 0.625%, 0.3125%, 0.15625%, 0.078125%, 0.0390625%, 0.01953125%. A blank control group and a positive control group were set, where the blank control group was a culture medium, and the positive control group was a culture medium added with 10% dimethylsulfoxide (DMSO) by mass fraction. The test groups, the blank control group, and the positive control group were brought into contact with test cells. The in vitro cytotoxicity was evaluated via cell growth inhibition assays to investigate the safety of the substrate as a human contact material. The test cells were human immortalized keratinocytes (HaCaTs).

TABLE 1
Cytotoxicity Test Results

| | | | Test | Test | Test | Test | Test | Test | Test | Test |
|---|---|---|---|---|---|---|---|---|---|---|
| | Blank | Positive | Group | Group | Group | Group | Group | Group | Group | Group |
| Group | Control | Control | 1 | 2 | 3 | 4 | 5 | 6 | 7 | 8 |
| Mean | 0.8846 | 0.2446 | 0.8339 | 0.8001 | 0.7761 | 0.7766 | 0.7732 | 0.7505 | 0.7720 | 0.7860 |
| SD | 2.64% | 0.38% | 0.52% | 0.72% | 0.72% | 0.74% | 0.72% | 1.01% | 0.66% | 0.53% |
| CV | 2.99% | 1.55% | 0.63% | 0.90% | 0.93% | 0.96% | 0.93% | 1.34% | 0.86% | 0.67% |
| Survival | 100% | 27.65% | 94.27% | 90.45% | 87.73% | 87.79% | 87.41% | 84.84% | 87.27% | 88.85% |
| rate | | | | | | | | | | |
| p-value | / | <0.0001 | 0.0154 | 0.0012 | 0.0003 | 0.0003 | 0.0002 | 0.0001 | 0.0002 | 0.0004 |

In the present disclosure, the cytotoxicity test results of the liquid composite prepared in Example 1 are shown in Table 1. The volume percentages of the liquid composite corresponding to test group 1, test group 2, test group 3, test group 4, test group 5, test group 6, test group 7, and test group 8 were 2.50%, 1.25%, 0.625%, 0.3125%, 0.15625%, 0.078125%, 0.0390625%, and 0.01953125%, respectively. The cell survival rates at the volume percentages of 2.50%, 1.25%, 0.625%, 0.3125%, 0.15625%, 0.078125%, 0.0390625%, and 0.01953125% in the test groups were 94.27%, 90.45%, 87.73%, 87.79%, 87.41%, 84.84%, 87.27%, and 88.85%, respectively, all greater than 70%. The tested liquid composite had no cytotoxicity. In the present disclosure, the succinate derivative prepared in Example 2 was subjected to the cytotoxicity test. The liquid composite was prepared according to the preparation method of Example 2, where the amount of the succinate derivative in the liquid composite was changed to 1 wt %. The liquid composite was added to a cell culture medium at a volume percentage of 2.50%, and the resulting cells had a survival rate of 92.51% without cytotoxicity.

### 2. Cell Proliferation Test

In the present disclosure, the liquid composite prepared in Example 1 was subjected to a cell proliferation test. The liquid composite of Example 1 was mixed with a culture to prepare 8 liquid composite test groups with different volume percentages: 2.50%, 1.25%, 0.625%, 0.3125%, 0.15625%, 0.078125%, 0.0390625%, 0.01953125%. A blank control group and a positive control group were set, where the blank control group was a culture medium, and the positive control group was a culture medium added with 10% DMSO by mass fraction. The test groups, the blank control group, and the positive control group were brought into contact with test cells. The proliferation effect on the cells was evaluated by calculating the cell proliferation rate on HSF cells. The test cells were human skin fibroblasts (HSFs).

TABLE 2
Cell Proliferation Test Results

| | | | Test | Test | Test | Test | Test | Test | Test | Test |
|---|---|---|---|---|---|---|---|---|---|---|
| | Blank | Positive | Group | Group | Group | Group | Group | Group | Group | Group |
| Group | Control | Control | 1 | 2 | 3 | 4 | 5 | 6 | 7 | 8 |
| Mean | 0.4411 | 0.5488 | 0.6203 | 0.6047 | 0.5569 | 0.531 | 0.5007 | 0.4998 | 0.4914 | 0.4274 |
| SD | 0.19% | 0.95% | 0.23% | 1.08% | 2.78% | 1.00% | 1.70% | 1.65% | 1.07% | 1.61% |
| CV | 0.43% | 1.73% | 0.37% | 1.78% | 4.99% | 1.89% | 3.40% | 3.30% | 2.17% | 3.77% |
| Survival | 100% | 124.42% | 140.64% | 137.11% | 126.25% | 120.40% | 113.51% | 113.31% | 111.40% | 96.89% |
| rate | | | | | | | | | | |
| p-value | / | 0 | 0 | 0 | 0.002 | 0.0001 | 0.0038 | 0.0036 | 0.0013 | 0.2175 |

In the present disclosure, the cell proliferation test results of the liquid composite prepared in Example 1 are shown in Table 2. The volume percentages of the liquid composite corresponding to test group 1, test group 2, test group 3, test group 4, test group 5, test group 6, test group 7, and test group 8 were 2.50%, 1.25%, 0.625%, 0.3125%, 0.15625%, 0.078125%, 0.0390625%, and 0.01953125%, respectively. The test groups had a significant cell proliferation promoting effect on HSFs at the volume percentages of 2.50%, 1.25%, 0.625%, 0.3125%, 0.15625%, 0.078125, and 0.0390625%.

### 3. Anti-Inflammatory Effect Test

In the present disclosure, the Luminex 200 liquid-phase suspension chip analyzer was employed to detect the expression of inflammatory cytokines after human fibroblasts were treated with test samples. In the present disclosure, human fibroblasts were treated with three different test samples, and cultured in a carbon dioxide incubator at 37° C. for 24 h, and then the supernatant was collected in a new plate for protein chip study to detect the expression levels of inflammatory cytokines. In the present disclosure, the above-mentioned test was divided into 4 groups, namely a negative control group, test group 1, test group 2, and test group 3, each group including 3 replicate wells. Water was added for the negative control group, while 0.5 vol % of the liquid composite prepared in Example 1 for the test group 1, 0.5 vol % of commercially available product 1 for the test group 2, and 0.5 vol % of commercially available product 2 for the test group 3. The commercially available product 1 was WINONA Revitalizing Repairing Essance, and the commercially available product 2 was HELENA RUBINSTEIN POWERCELL SKINMUNITY.

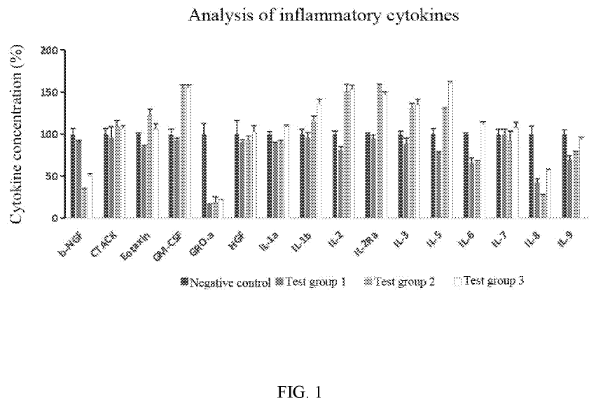

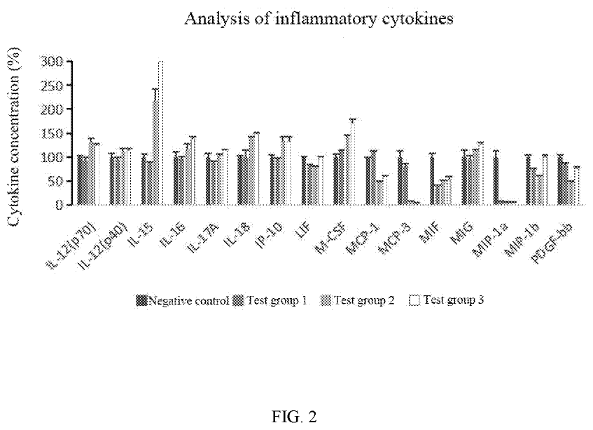

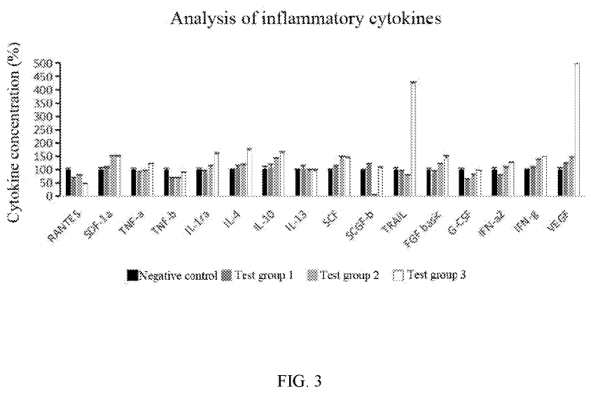

In this study, the expression of a total of 48 inflammatory cytokines was detected, with the results being shown in FIG. 1 to FIG. 3. The significance analysis of the results of 8 inflammatory cytokines was conducted, with the results being shown in

TABLE 3
Statistical Table of Significance of Inflammatory Cytokines

| | Inflammatory cytokine | | | | | | | |
|---|---|---|---|---|---|---|---|---|
| Group | GRO-a↓ | IL-3↓ | IL-5↓ | IL--6↓ | IL-17A↓ | MIF↓ | TNF-a↓ | TNF-b↓ |
| Test | ** | * | * | * | * | ** | * | * |
| group 1 | | | | | | | | |
| Test | ** | | | * | | * | | * |
| group 2 | | | | | | | | |
| Test | ** | | | | | * | | |
| group 3 | | | | | | | | |

The statistics of significance of the 8 inflammatory cytokines in the present disclosure are shown in Table 3, in which the test group 1, the test group 2, and the test group 3 all exhibited significant down-regulation capability for the expression of CRO-alpha inflammatory cytokine. Through an in-depth comparative study of three skin care essence products, it was found that the test group 1 exhibited the most significant inhibitory effect on inflammation at 0.5 vol %.

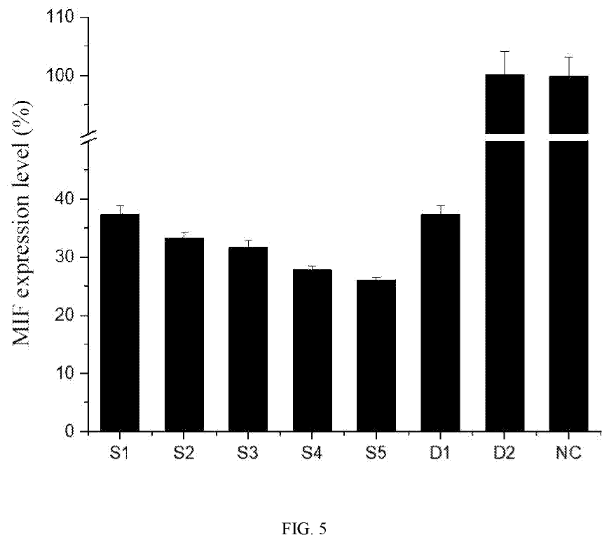

In the present disclosure, the liquid composites prepared in Examples 1-5 and Comparative Examples 1-2 were subjected to an anti-inflammatory test. A negative control group, which was water, was set. Comparative analysis was performed on the expression of the inflammatory cytokines of MIF, with the results being as shown in FIG. 5, where S1 represents Example 1, S2 represents Example 2, S3 represents Example 3, S4 represents Example 4, S5 represents Example 5, D1 represents Comparative Example 1, D2 represents Comparative Example 2, and NC represents the negative control group. In the present disclosure, an active ingredient, an excipient, and water were utilized to prepare a liquid composite. The active ingredient included at least an active substance. The excipient included a thickener, a humectant, and a preservative. The active substance might be at least one of acetyl hexapeptide-8, oligopeptide-1, dipeptide diaminobutyroyl benzylamide diacetate, carnosine, palmitoyl tripeptide-1, ACE liposome, and bifida ferment lysate. The liquid composite obtained by co-formulating the active ingredient and the excipients, exhibited good anti-inflammatory effect. An active agent might further be added to the active ingredient. The active agent of the present disclosure may be a succinate derivative which was prepared from 2-aminoethyl hydrogen sulfate and a maleate. The maleate was prepared from trimethylolpropane and maleic anhydride. The liquid composite obtained by compounding the succinate derivative with the active substance and the excipients exhibited better anti-inflammatory capability and better effect of reducing the relative expression of the MIF gene. With the increase of the amount of the succinate derivative used, the resulting liquid composite exhibited increased anti-inflammatory capability, and could reduce the relative expression of the MIF gene. When the amount of the succinate derivative used in the active agent was too low, the anti-inflammatory capability of the liquid composite could not be enhanced, and the relative expression of the MIF gene could not be reduced. Propane-1,2-diol alginate could also be added to the active agent of the present disclosure, and when the propane-1,2-diol alginate and the succinate derivative were used as the active agents together with the active substance and the excipients, the resulting liquid composite exhibited better anti-inflammatory capability and better effect of reducing the relative expression of the MIF gene. If no active substance was used in the liquid composite, but only the active agent was added, the resulting liquid composite exhibited poor anti-inflammatory capability and could not significantly reduce the relative expression of the MIF gene.

### 4. Quantitative Gene Expression Test for Anti-Aging Effect

In the present disclosure, Luminex 200 suspension chip multi-detection system technology was used in combination with the QuantiGene Plex (QGP) method to detect and analyze the effects of three test samples on gene expression in human fibroblasts. In the present disclosure, three different test samples were administered to HFF cells, and plated at a density of 8×10⁴ cells per well in a 24-well plate in a volume of 0.5 ml per well. The cell plates were incubated for 24 h in an incubator.

In the present disclosure, the above-mentioned test was divided into 4 groups, namely a negative control group, test group 1, test group 4, and test group 5, each group including 3 replicate wells. Water was added for the negative control group, while 0.5 vol % of the liquid composite prepared in Example 1 for the test group 1, 0.5 vol % of commercially available product 3 for the test group 4, and 0.5 vol % of commercially available product 4 for the test group 5. The commercially available product 1 was WINONA Revitalizing Repairing Essance, and the commercially available product 2 was HELENA RUBINSTEIN POWERCELL SKINMUNITY. Their contents were each 0.5 vol %. After 24 h of cultivation, the supernatant was aspirated off and the cells were lysed by adding 0.2 ml of cell lysis solution to the well plates containing HFF cells. The lysed cells were preserved at −80° C. The commercially available product 4 was a certain essence of a brand name starting with W, and the commercially available product 5 was HELENA RUBINSTEIN POWERCELL SKINMUNITY.

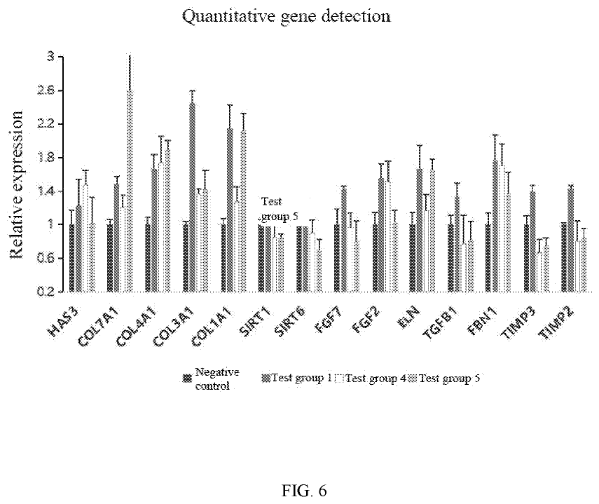

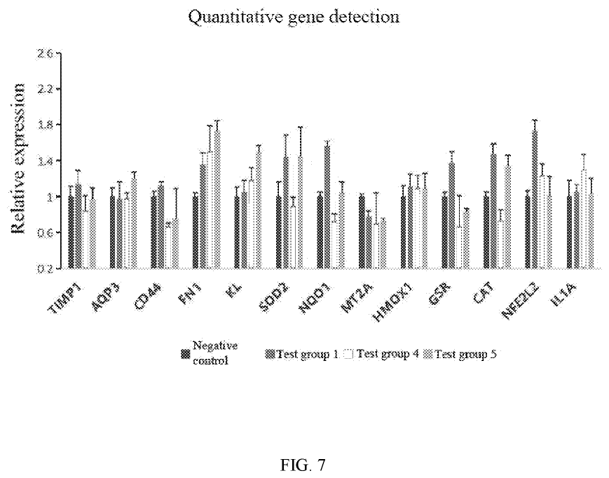

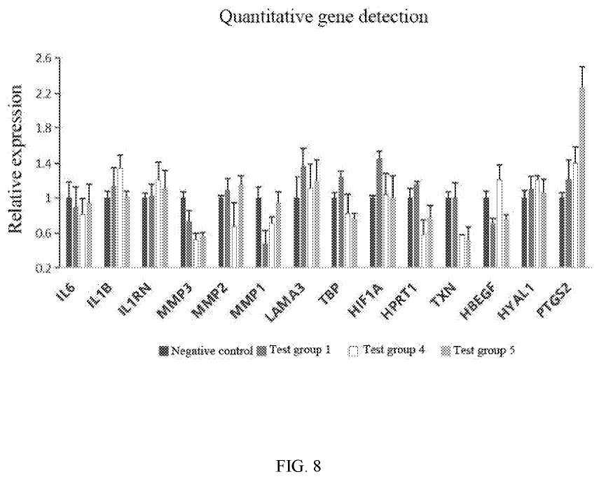

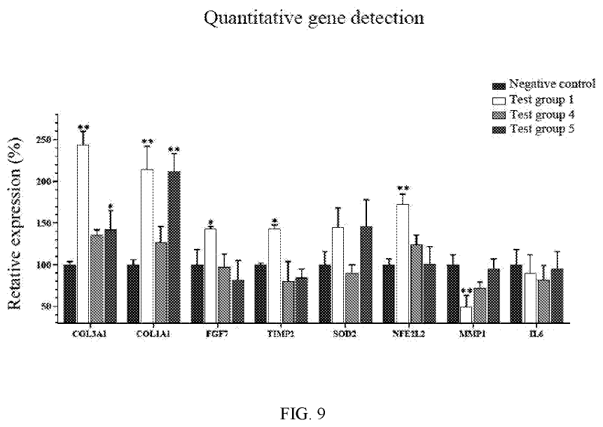

In the present disclosure, the expression of a total of 41 genes was detected, and the cells were treated with three test samples. The whole-genome results are shown in FIG. 6 to FIG. 8. Eight of the genes, COL3A1, COL1A1, FGF7, TIMP2, SOD2, NFE2L2, MMP1, and IL6, were analyzed for significance in the study, with the results being shown in FIG. 9 and Table 4.

TABLE 4
Significance Analysis Results of Seven Genes

| | COL3A1 | COL1A1 | FGF7 | TIMP2 | SOD2 | NFE2L2 | MMPI | IL6 |
|---|---|---|---|---|---|---|---|---|
| Test group 1 | ** | ** | * | * | | ** | ** | |
| Test group 4 | | | | | | | | |
| Test group 5 | * | ** | | | | | | |

The 0.5 vol % test group 1 could significantly up-regulate the relative expression of the genes COL3A1, COL1A1, and NFE2L2 (p<0.01), significantly up-regulate the relative expression of the genes FGF7 and TIMP2 (p<0.05), and also significantly down-regulate the relative expression of the gene MMP1 (p<0.01). The 0.5 vol % test group 5 could significantly up-regulate the relative expression of the gene COL1A1 (p<0.01).

In conclusion, among the test group 1, the test group 4, and the test group 5, the test group 1 exhibited better effect on the regulation of aging genes, and exhibited significant anti-aging capability.

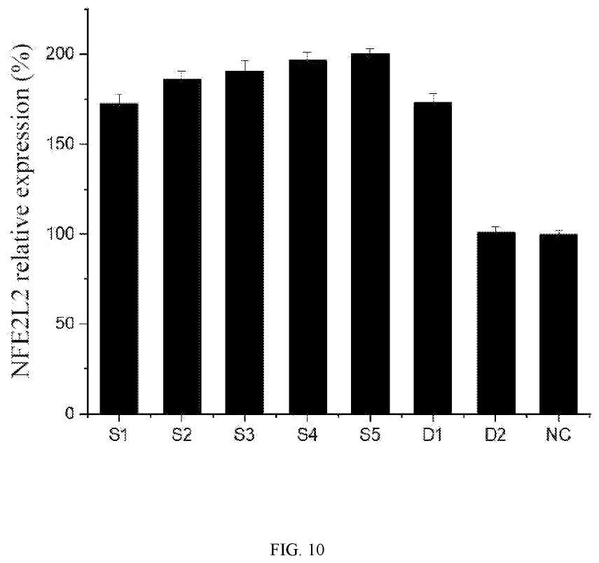

In the present disclosure, the liquid composite prepared in Examples 1-5 and Comparative Examples 1-2 were subjected to an anti-aging test. A negative control group, which was water, was set. Comparative analysis was performed on the relative expression of the gene NFE2L2, with the results being as shown in FIG. 10, where S1 represents Example 1, S2 represents Example 2, S3 represents Example 3, S4 represents Example 4, S5 represents Example 5, D1 represents Comparative Example 1, D2 represents Comparative Example 2, and NC represents the negative control group. In the present disclosure, an active ingredient, an excipient, and water were utilized to prepare a liquid composite. The active ingredient included at least an active substance. The excipient included a thickener, a humectant, and a preservative. The active substance might be at least one of acetyl hexapeptide-8, oligopeptide-1, dipeptide diaminobutyroyl benzylamide diacetate, carnosine, palmitoyl tripeptide-1, ACE liposome, and bifida ferment lysate. The liquid composite obtained by co-formulating the active ingredient and the excipients, exhibited good anti-aging effect. An active agent might further be added to the active ingredient. The active agent of the present disclosure may be a succinate derivative which was prepared from 2-aminoethyl hydrogen sulfate and a maleate. The maleate was prepared from trimethylolpropane and maleic anhydride. The liquid composite obtained by compounding the succinate derivative with the active substance and the excipients exhibited better anti-aging capability and better effect of increasing the relative expression of the gene NFE2L2. With the increase of the amount of the succinate derivative used, the resulting liquid composite exhibited increased anti-aging capability, and could increase the relative expression of the gene NFE2L2. When the amount of the succinate derivative used in the active agent was too low, the anti-aging capability of the liquid composite could not be enhanced, and the relative expression of the gene NFE2L2 could not be increased. Propane-1,2-diol alginate could also be added to the active agent of the present disclosure, and when the propane-1,2-diol alginate and the succinate derivative were used as the active agents together with the active substance and the excipients, the resulting liquid composite exhibited better anti-aging capability and better effect of increasing the relative expression of the gene NFE2L2. If no active substance was used in the liquid composite, but only the active agent was added, the resulting liquid composite exhibited poor anti-aging capability and could not significantly increase the relative expression of the gene NFE2L2.

### 5. Human Skin Patch Test

Using the 2015 Safety and Technical Standards for Cosmetics as the reference criteria, the liquid composite prepared in Example 1 was used as the test sample for skin irritation evaluation of a skin retention product. The test method employed was the skin patch test, which was conducted on 30 randomly selected subjects aged 18-60 years.

Test Method: The test sample (0.025 g) was placed into a patch tester. The patch tester containing the test sample was then applied to the flexor surface of the subject's forearm using a non-irritating fabric-based adhesive tape, and was gently pressed with the palm to ensure uniform adhesion to the skin surface. After 24 hours, the patch tester was removed. Skin reactions were observed 30 minutes after removal, once the pressure marks had disappeared. If the result was negative, observations were repeated 24 hours and 48 hours after the patch test.

### Evaluation Criteria:

Level 0: Negative reaction.

Level 1: Doubtful reaction; and faint erythema only.

Level 2: Weak positive reaction; erythema, infiltration, edema, and possibly papules.

Level 3: Strong positive reaction; erythema, infiltration, edema, and possibly papules; and reaction may extend beyond the test area.

Level 4: Very strong positive reaction; marked erythema, severe infiltration, edema, and coalescing vesicles; and reaction extends beyond the test area.

Test Results: All subjects exhibited negative skin reactions.

The above test results showed that the liquid composite prepared in the present disclosure was mild, non-irritating, and safe for use as a skin retention product.

6. Anti-Inflammatory Effect Test after Medical Aesthetic Procedures

Thirty volunteers who underwent mild medical aesthetic procedures, such as intense pulsed light and hydrating injection, were recruited, aged 20-50 years. The liquid composite prepared in Example 1 was used as the test product 3 days after the procedures. After cleansing the face, the test product was evenly applied to the face. It was used continuously for 28 days. Skin conditions were observed on day 0, day 14, and day 28.

### Evaluation Indicators:

6.1. TEWL Value Detection: TEWL refers to trans epidermal water loss, which is inversely related to the stratum corneum hydration level. The lower the TEWL value, the higher the stratum corneum hydration level, and the better the skin barrier function. Conversely, a higher value indicates poorer barrier function.

TEWL values: health status: (0-15), sub-health status: (15-25), and unhealthy status: (>25).

TABLE 5
TEWL Value Detection results

| | | | TEWL [g/m²/h] | |
|---|---|---|---|---|
| | | Sample Size | Average | Improvement Rate |
| Sample group | D 0 | 30 | 15.43 | — |
| | D 14 | 30 | 15.06 | 2.40% |
| | D 28 | 30 | 13.54 | 12.25% |

After 28 days of using the test product, the TEWL of the subjects showed a very significant decrease. This indicated a marked improvement in TEWL compared to the initial value after continuous use of the test product, with an improvement rate of 12.25%.

6.2. Hemoglobin Value Detection: The hemoglobin content in the skin was determined by measuring the amount of light of a specific wavelength reflected from the human skin. The lower the hemoglobin content value, the lighter the redness.

TABLE 6
Hemoglobin Value Detection Results

| | | | Hemoglobin [μmol/L] | |
|---|---|---|---|---|
| | | Sample Size | Average | Improvement Rate |
| Sample group | D 0 | 30 | 343.66 | — |
| | D 14 | 30 | 339.59 | 1.18% |
| | D 28 | 30 | 322.87 | 6.05% |

After 28 days of using the test product, the hemoglobin content of the subject showed a very significant decrease. This indicated an improvement in hemoglobin content compared to the initial value after continuous use of the test product, with an improvement rate of 6.05%.

### 6.3. Feedback on Adverse Reactions During Use

All 30 subjects used the test product as required within the specified time, and no adverse reactions were observed.

The embodiments and/or implementations described above are only intended to illustrate the preferred embodiments and/or implementations for carrying out the technology of the present disclosure, rather than to limit the implementations of the technology of the present disclosure in any form. Any person skilled in the art, without departing from the scope of the technical means disclosed herein, may make appropriate modifications or changes to other equivalent embodiments, but these should still be regarded as technologies or embodiments substantially the same as the present disclosure.

Specific embodiments are used for illustrating principles and implementations of the present disclosure herein. The description of the embodiments above is only used for helping understand the method and its core concept of the present disclosure. The foregoing are merely the preferred implementations of the present disclosure. It should be pointed out that since the text expression is limited while there are objectively infinite specific structures, those of ordinary skill in the art can also make several improvements, embellishments or changes without departing from the principles of the present disclosure, or can combine the foregoing technical features in appropriate ways. These improvements, embellishments, changes or combinations, or those that directly apply the concepts and technical solutions of the present disclosure to other occasions without improvement, shall be regarded as falling within the protection scope of the present disclosure.

What is claimed is:

1. A liquid composite, comprising: an active ingredient and water, wherein a content of the active ingredient in the liquid composite is 0.003-6.0 wt %; the active ingredient comprises an active substance that comprises at least one of acetyl hexapeptide-8, oligopeptide-1, dipeptide diaminobutyroyl benzylamide diacetate, carnosine, palmitoyl tripeptide-1, palmitoyl tripeptide-5, ACE liposome, and bifida ferment lysate;
the active ingredient further comprises an active agent that comprises a succinate derivative and propane-1,2-diol alginate;
the succinate derivative is prepared by: adding sodium hydroxide and 2-aminoethyl hydrogen sulfate to deionized water, and mixing with stirring; then adding a maleate and ethanol at 30-50° C. and reacting for 2-8 h; after the reaction is completed, cooling to form a precipitate; and carrying out filtration, washing, and distillation under reduced pressure to obtain the succinate derivative, wherein the sodium hydroxide is used in an amount of 30-50 wt % of the deionized water, while the 2-aminoethyl hydrogen sulfate is used in an amount of 40-60 wt % of the deionized water and in an amount of 20-30 wt % of the maleate, and absolute ethanol is used in an amount of 100-200 wt % of the maleate;
the maleate is prepared by: adding trimethylolpropane to maleic anhydride at 60-80° C., and reacting at 85-95° C. for 2-8 h; and after the reaction is completed, obtaining the maleate, wherein the trimethylolpropane is used in an amount of 50-150 wt % of the maleic anhydride;
a content of the active agent in the liquid composite is 0.003-1.0 wt %; or, a content of the active substance in the liquid composite is 0.003-5.0 wt %.

2. The liquid composite according to claim 1, further comprising a thickener that comprises at least one of carbomer, xanthan gum, and sclerotium gum, wherein a content of the thickener in the liquid composite is 0.01-1.0 wt %.

3. The liquid composite according to claim 1, further comprising a humectant that comprises at least one of glycerin, 1,2-pentanediol, 1,2-hexanediol, dipropylene glycol, carboxymethyl cellulose, and chitin, wherein a content of the humectant in the liquid composite is 1-20 wt %.

4. The liquid composite according to claim 1, further comprising a preservative that is a raspberry ketone system, wherein a content of the preservative in the liquid composite is 0.1-1.0 wt %.
